(19) United States
(12) Patent Application Publication
(10) Pub. No.: US 2016/0193426 A1
(43) Pub. Date: Jul. 7, 2016

(54) DRIVE ASSEMBLY FOR A DRUG DELIVERY DEVICE

(71) Applicant: Sanofi, Paris (FR)

(72) Inventor: Michael BAINTON, Kineton (GB)

(21) Appl. No.: 14/916,080

(22) PCT Filed: Sep. 3, 2014
(86) PCT No.: PCT/EP2014/068658

§ 371 Date: Mar. 2, 2016

(30) Foreign Application Priority Data
Sep. 3, 2013 (EP) 13182766.9

(51) Int. Cl. A61M 5/315

(52) U.S. Cl. CPC A61M 5/31566; A61M 5/31515

(57) ABSTRACT

The present disclosure concerns a drive assembly (201) for a drug delivery device. The drive assembly comprises a piston rod (214) comprising a bearing (217) and a safety member (253) that is configured to prevent a movement of the bearing (217) of the piston rod (214) when the drive assembly (201) is damaged.

The present invention concerns a drive assembly for a drug delivery device.

Drug delivery devices are generally known for the administration of a medicinal product, for example insulin or heparin, but also for other medicinal products, in particular for self-administration by a patient. A drug delivery device may be configured as a pen-type injector which may dispense a variable dose of a fluid medicinal product. However, the drug delivery device may also deliver a pre-set dose of a medicinal product.

It is an object of the present disclosure to provide a drive assembly for a drug delivery device which helps to improve the usability and the safety of the drug delivery device.

This object is solved by the drive assembly according to present claim 1.

According to the present disclosure, a drive assembly for a drug delivery device is provided which comprises a piston rod comprising a bearing and a safety member. The safety member is configured to prevent a movement of the bearing of the piston rod when the drive assembly is damaged.

The drive assembly being damaged may correspond to one or more elements of the drive assembly being damaged. In particular, the piston rod may be damaged. When the drive assembly is damaged, it may no longer be possible to carry out a correct dose setting operation and/or a correct dose dispense operation.

When the drive assembly is damaged, this may result in an unintentional dose dispense operation or in dispensing of a false amount of a drug in a dose dispense operation. As the safety member prevents any further movement of the piston rod when the drive assembly is damaged, the safety member may prevent an unintentional dispensing of the medicinal product. Additionally or alternatively, the safety member may prevent that the wrong amount of the medicinal product is dispensed in a dose dispensing operation. Thereby, the safety member protects the patient from a false treatment with the wrong dose.

When the drive assembly is damaged, this may result in the piston rod expelling the complete medicinal product of the cartridge. As the safety member prevents a movement of the piston rod in this case, the safety member may protect a user from unintentionally injecting the complete medicinal product of the cartridge. This significantly increases the usability and the safety of the drive assembly. For example, due to a faulty use, the drive assembly may be damaged during a dose dispense operation wherein a patient has injected a needle of the drug delivery device. In this case, the safety member may prevent the complete dose of the medicament from being delivered to the patient. Otherwise, this would result in a false dosing which could have significant consequences for the health of the patient.

When the piston rod is prevented from moving, a dose dispensing operation may no longer be possible. Accordingly, the patient immediately realises that the drive assembly of the drug delivery device must have been damaged. Thus, the safety member helps to alert a patient of a damaged drive assembly.

The piston rod may be tensed in an undamaged state of the drive assembly. Further, the drive assembly being damaged may result in the tension of the piston rod being relieved.

Without the safety member, a relief of the tension of the piston rod may further result in dispensing of the complete medicinal product of a cartridge of the drug delivery device. However, the safety member may be configured to prevent a movement of the piston rod when the tension of the piston rod is relieved. In particular, the safety member may be configured such that in case of a relief of the tension of the piston rod, the safety member prevents any further movement of the piston rod.

The tension of the piston rod may be relieved, if the piston rod breaks or is detached at one of its ends.

The safety member may comprise a first safety member part and a second safety member part, wherein the second safety member part may be engageable to the first safety member part. A movement of the piston rod may be prevented when the first safety member part and the second safety member part are engaged with each other.

Accordingly, in the normal use of the drug delivery device, i.e. when the drive assembly is undamaged, the first and the second safety member parts may not be engaged with each other. The drive assembly may be constructed such that a damage of the drive assembly automatically results in an engagement of the first and the second safety member parts.

This provides the advantage that the safety member does not interfere with the use of the drug delivery device when the drive assembly is undamaged. Instead, when the drive assembly is undamaged, the safety member parts may not be engaged with each other and, further, may not exert a force on other elements of the drive assembly such that the safety member does not increase the forces required to carry out a dose setting operation or a dose dispense operation.

The first safety member part may comprise a strap. The strap may run parallel to the piston rod. The strap may be attached to one end of the piston rod.

The second safety member part may comprise a spring arm. The spring arm may be attached to a housing of the drug delivery device.

The second safety member part may be pretensioned in a direction towards the first safety member part. In an undamaged drive assembly, the second safety member may abut the piston rod wherein the tension of the piston rod may be sufficient to resist deformation of the piston rod under the action of a transverse force applied to the piston rod by the second safety member part, thereby preventing an engagement of the second safety member part with the first safety member part. However, when the drive assembly is damaged, the piston rod may lose its tension and the tension of the second safety member part may overcome the reduced tension of the piston rod, thereby engaging the second safety member part with the first safety member part.

The safety member may comprise a spacer member that is adapted to prevent an engagement of the first and the second safety member part when the drive assembly is undamaged.

In particular, the spacer member may be integrally formed with the spring arm. The spacer member may be constructed such that it is enabled to detect a damage of the drive assembly. A damaged drive assembly may trigger a movement of the spacer member wherein, due to this movement, the first and the second safety member parts are engaged with each other.

The safety member may abut the piston rod. In particular, the piston rod may be tensed when the drive assembly is undamaged. The tension of the piston rod may prevent a movement of the safety member and may, thereby, prevent an engagement of the first and the second safety member parts. Moreover, the piston rod may lose its tension when the drive assembly is damaged such that the spacer member is enabled to move the piston rod, thereby engaging the first and the second safety member parts.

The drive assembly may further comprise a spring member wherein the drive assembly may be configured such that a relaxation of the spring member moves the piston rod. Further, an engagement of the safety member parts may prevent the relaxation of the spring member.

During the assembly of the drive assembly, the spring member may be compressed such that it is capable of delivering all of the required doses from a cartridge when allowed to release. In a dose dispense operation, the spring member may be allowed to release its compression stepwise. Further, the safety member may be configured to prevent the spring member from releasing its compression completely all at once and thereby from expelling the complete medicinal product when the drive assembly is damaged.

The spring member may be tensed between a first spring seat and a second spring seat. The first safety member part may be connected to the first spring seat. The second safety member part may be connected to the second spring seat.

The first spring seat may be formed by the piston rod. In particular, the first spring seat may be formed by a bearing arranged at the distal end of the piston rod. Further, the second spring seat may be formed by a part of the housing of the drug delivery device.

Hereby, the terms “distal” and “proximal” shall be defined as follows. In an assembled drug delivery device, the distal end of an element of the drug delivery device is defined as the end of the element which is closest to a dispensing end of the drug delivery device. In an assembled drug delivery device, the proximal end of an element of the drug delivery device is defined as the end of the element which is furthest away from the dispensing end of the drug delivery device. Moreover, a distal direction is defined as a direction towards the distal end and a proximal direction is defined as a direction towards the proximal end.

An engagement of the first safety member part and the second safety member part may prevent a movement of the first spring seat relative to the second spring seat. In particular, it may prevent a movement of the first spring seat in a direction away from the second spring seat. Thereby, a further relaxation of the spring member may be prevented.

As a relaxation of the spring member may be prevented by the safety member, this may result in a dose dispense operation being prevented. In particular, the drive assembly may be constructed such that a dose is dispensed when the spring member relaxes.

The piston rod may form the first spring seat. The part of the housing to which the second safety member part is attached may form the second spring seat.

The first safety member part may comprise a first engagement member. The first engagement member may comprise teeth arranged at the surface of the first safety member part. Further, the second safety member part may comprise a second engagement member. The second engagement member may comprise a protrusion which is adapted to engage with the first engagement member.

The piston rod may be flexible. In particular, the piston rod may be constructed such that it is wound around other elements of the drive assembly, e.g. around a pinion gear of a drive control member. This allows constructing a compact drive assembly which requires only a small space.

The safety member may be configured to mechanically detect a damage of the drive assembly. In particular, the safety member may be configured to prevent a movement of the bearing of the piston rod when the safety member detects in a mechanical manner that the drive assembly is damaged. The safety member may, in particular, be configured to detect a mechanical engagement of a first safety member part and a second safety member part. Thereby, the mechanical engagement of the first safety member part and the second safety member part may correspond to a damage of the drive assembly. In particular, the safety member may be constructed such that the first and the second safety member parts are enabled to mechanically engage with each other only when the drive assembly is damaged. Thus, from a mechanical engagement of the first and the second safety member parts, the safety member may deduce that the drive assembly is damaged.

Further, the drive assembly may be a manually operable assembly. Thus, the drive assembly may not comprise a motor or an electronic component. Instead, an operation of the drive assembly may be carried out solely based on the forces exerted by a user operating the drive assembly and not assisted by a motor or an electronic component.

The drive assembly may be a purely mechanical assembly, not an electro-mechanical assembly.

A second aspect of the present disclosure concerns a drug delivery device comprising the drive assembly. In particular, the drive assembly may be the drive assembly disclosed above such that every structural and functional feature disclosed with respect to that drive assembly may also be present in the drug delivery device.

The drug delivery device may further comprise a cartridge comprising a piston wherein the drive assembly is adapted to provide a force on the piston such that the piston is moved in the distal direction further into the cartridge. Thereby, a medicinal product may be expelled from the cartridge.

The drug delivery device may be configured as a pen-type injector which may dispense a variable dose of a fluid medicinal product. However, the drug delivery device may also deliver a pre-set dose of a medicinal product. Further, the drug delivery device may be a disposable device. Accordingly, the drug delivery device may not be operable after the last dose of the medicinal product has been expelled from the cartridge.

The term “medicinal product”, as used herein, preferably means a pharmaceutical formulation containing at least one pharmaceutically active compound, wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound, wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis, wherein in a further embodiment the pharmaceutically active compound comprises at least one peptide for the treatment and/or prophylaxis of diabetes mellitus or complications associated with diabetes mellitus such as diabetic retinopathy, wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-idecanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro- Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(02)25, Asp28] Exendin-4(1-39),

des Pro36 [Trp(02)25, IsoAsp28] Exendin-4(1-39),

des Pro36 [Met(0)14 Trp(02)25, Asp28] Exendin-4(1-39),

des Pro36 [Met(0)14 Trp(02)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(02)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two 13 sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; a and y contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts.

Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted Cl C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

In the following, the disclosed devices and methods are described in further detail with reference to the drawings, wherein

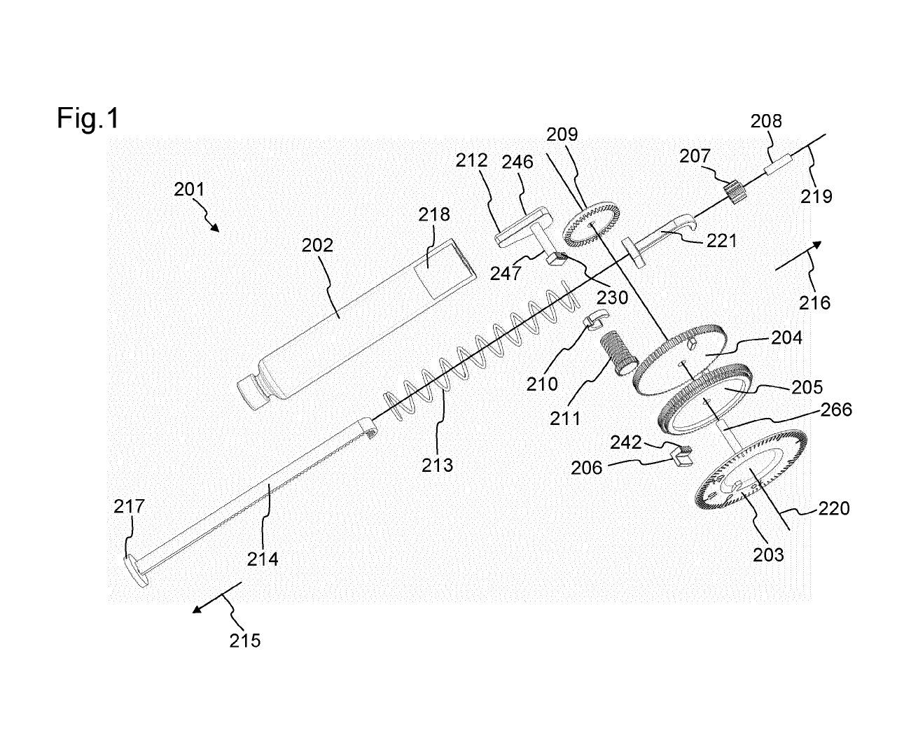

FIG. 1 shows an exploded view of a drive assembly for a drug delivery device,

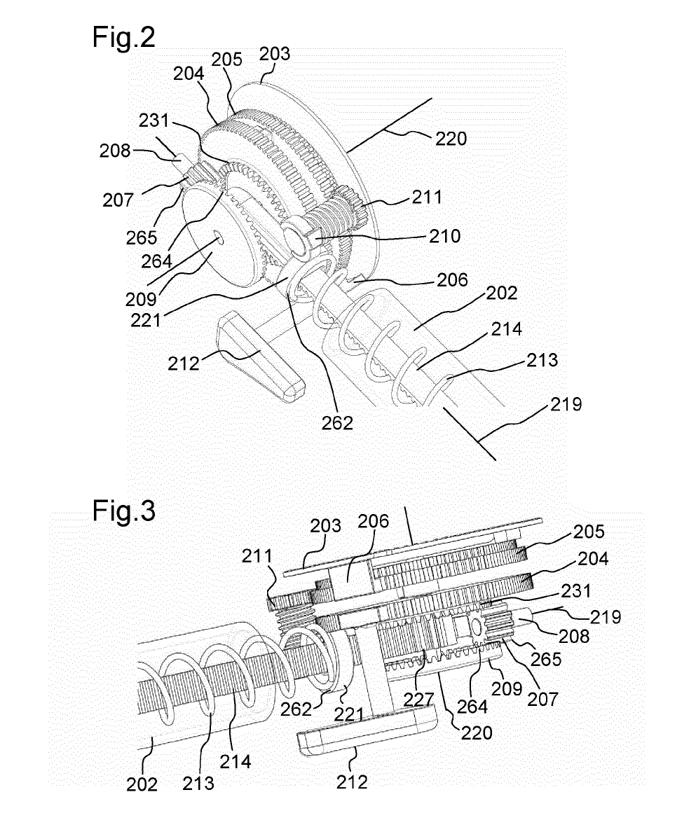

FIG. 2 shows a perspective view of the assembled drive assembly shown in FIG. 1,

FIG. 3 shows another perspective view of the assembled drive assembly shown in FIG. 1,

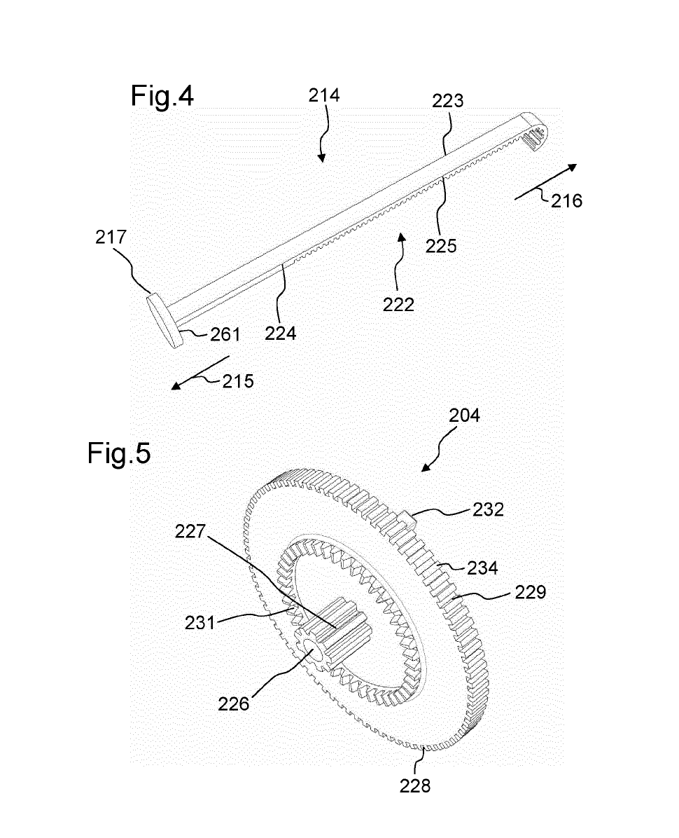

FIG. 4 shows a perspective view of a piston rod,

FIG. 5 shows a perspective view of a drive control member,

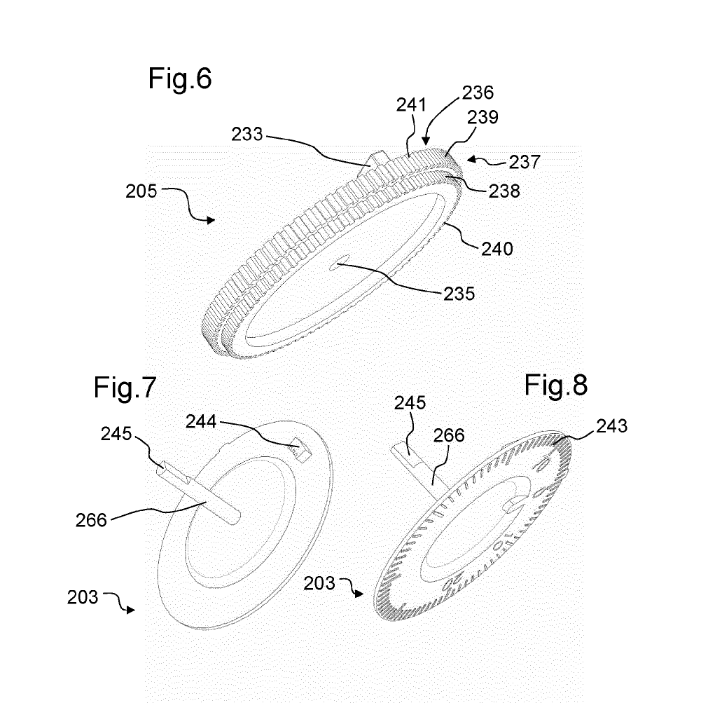

FIG. 6 shows a perspective view of a secondary drive control member,

FIG. 7 shows a perspective view of a dose setting member,

FIG. 8 shows another perspective view of the dose setting member,

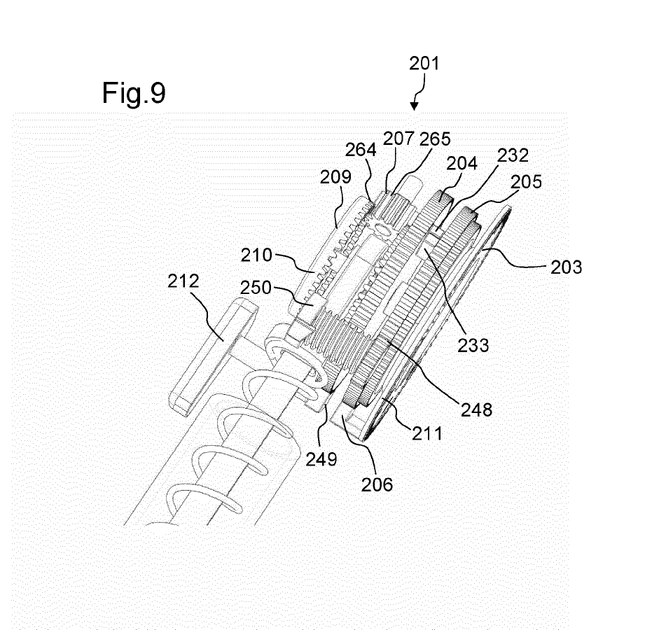

FIG. 9 show the drive assembly of FIGS. 1 to 3 in a rest state,

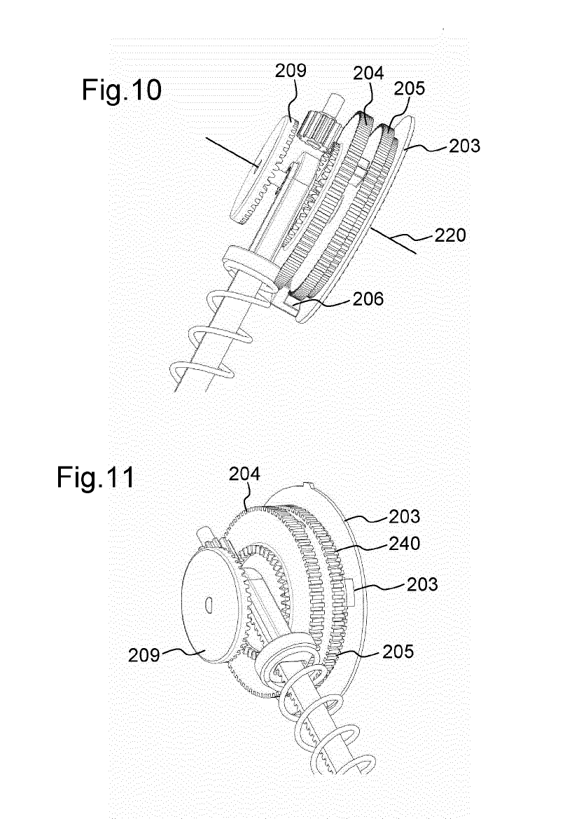

FIG. 10 shows the drive assembly in a ready-to-set state,

FIG. 11 shows the drive assembly in a ready-to-set state from a different perspective,

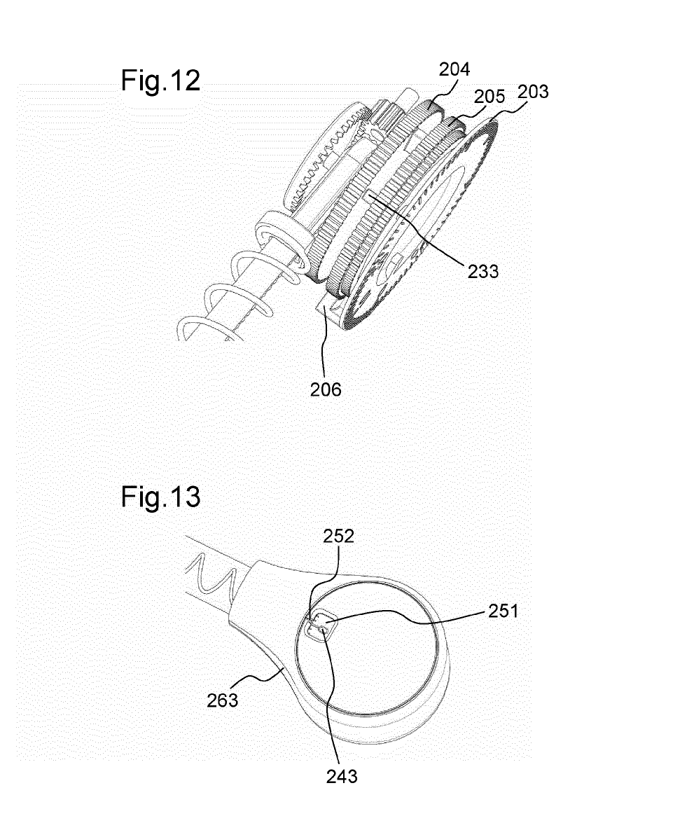

FIG. 12 shows the drive assembly in a dose-set state,

FIG. 13 shows a part of the housing comprising a window,

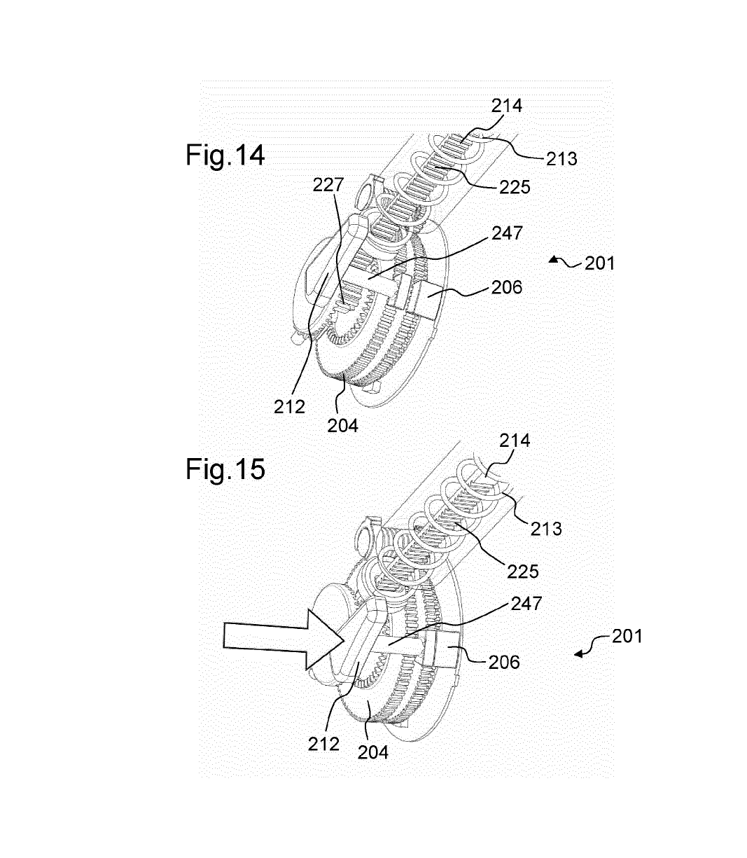

FIG. 14 shows the drive assembly after a dose setting operation is completed,

FIG. 15 shows the drive assembly during initiation of a dose dispensing operation,

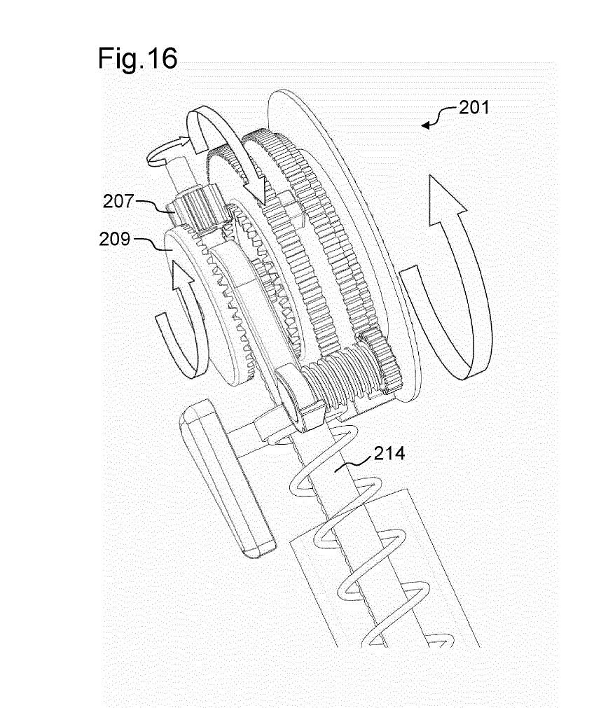

FIG. 16 shows the drive assembly during a dose dispensing operation,

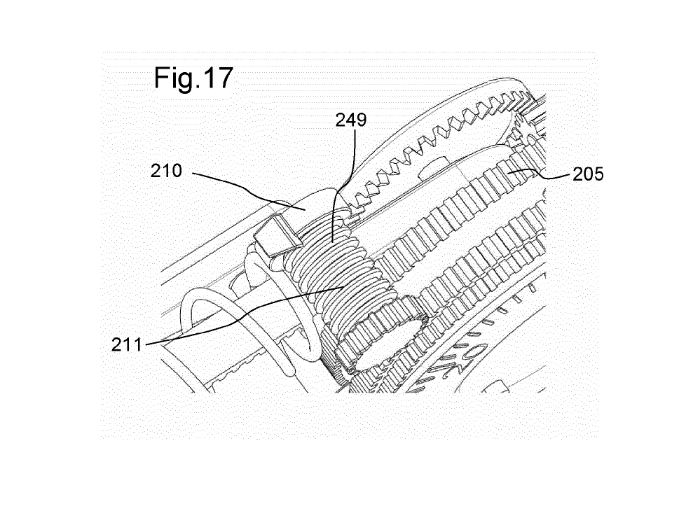

FIG. 17 shows a last dose lockout assembly of the drive assembly,

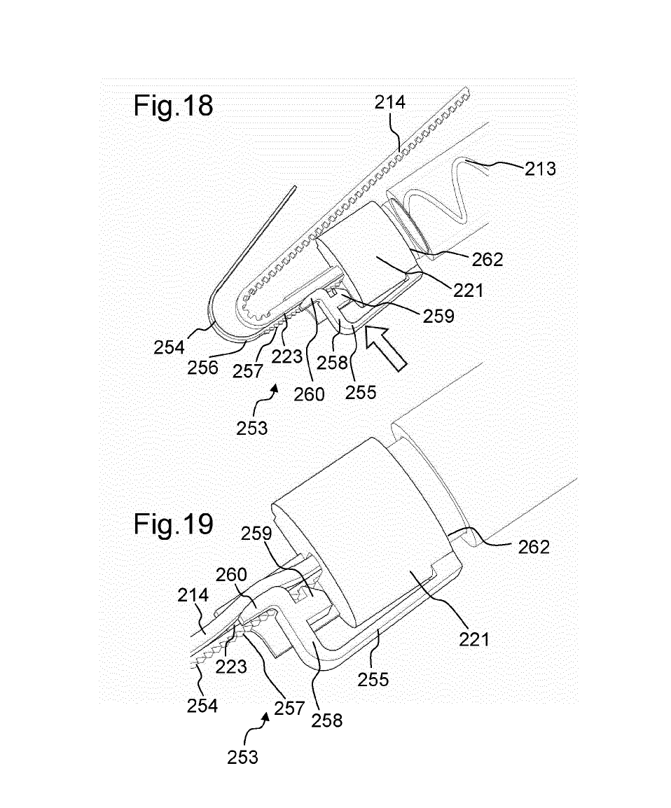

FIG. 18 shows the drive assembly comprising a safety member wherein the drive assembly is undamaged, and

FIG. 19 shows the drive assembly comprising the safety member wherein the drive assembly is damaged.

FIG. 1 shows an exploded view of a drive assembly 201 for a drug delivery device. The drive assembly 201 can be operated to deliver variable doses of a medicinal product from a cartridge 202, via a needle (not shown).

The drive assembly 201 comprises a dose setting member 203, a drive control member 204, a secondary drive control member 205, a drive control member stop 206, a reversing member 207, a reversing member shaft 208, a coupling member 209, a last dose stop member 210, a last dose stop drive member 211, an actuator 212, a spring member 213 and a piston rod 214. The components of the drive assembly 201 will be discussed in detail in the following. The drive assembly 201 is configured to move a piston 218 further into the cartridge 202 in a distal direction 215.

The piston rod 214 comprises a bearing 217 arranged at the distal end of the piston rod 214. The bearing 217 is adapted to provide a force on the piston 218 arranged in the cartridge 202 such that the piston 218 is moved in the distal direction 215 further into the cartridge 202. Thereby, a medicinal product is expelled from the cartridge 202.

The drive assembly 201 comprises a main axis 219. The main axis 219 of the drive assembly 201 corresponds to a longitudinal axis of the cartridge 202. The piston rod 214, the spring member 213, the reversing member 207 and the reversing member shaft 208 are located on the main axis 219 of the drive assembly 201.

Further, the drive assembly 201 defines a second axis 220. The second axis 220 is perpendicular to the main axis 219. In particular, the second axis 220 is defined by a shaft 266 of the dose setting member 203. In the drive assembly 201, the dose setting member 203, the secondary drive control member 205, the drive control member 204 and the coupling member 209 are arranged coaxially on the second axis 220.

The drive assembly 201 is configured to be located in a housing of the drug delivery device. In FIG. 1, the housing is not fully represented for clarity. However, in FIG. 1, a housing part 221 is shown.

Moreover, the drive assembly 201 may comprise a safety member which is not shown in FIG. 1. The safety member may be configured to prevent a movement of the piston rod 214 when the drive assembly 201 is damaged. The safety member will be discussed in detail later on.

FIGS. 2 and 3 show perspective views of the assembled drive assembly 201. In particular, the main axis 219 and the second axis 220 are shown in FIGS. 2 and 3.

FIG. 4 shows the piston rod 214. The piston rod 214 comprises the bearing 217 at its distal end. The bearing 217 is integrally formed with the piston rod 214. In particular, the bearing 217 forms a first spring seat 261. In the assembled drive assembly 201, one end of the spring member 213 abuts the first spring seat 261.

Moreover, the piston rod 214 is flexible such that it can be wound around other elements of the drive assembly 201. In particular, as shown in FIG. 3, the piston rod 214 is wound around an inner small diameter pinion gear 227 of the drive control member 204.

The piston rod 214 comprises a main part 222 extending in the proximal direction 216 from the bearing 217. The main part 222 has an upper main surface 223 and a lower main surface 224. In the assembled drive assembly 201, as shown in FIGS. 2 and 3, the lower main surface 224 of the piston rod 214 faces towards the inner small diameter pinion gear 227 of the drive control member 204. Further, in the assembled drive assembly 201, the upper main surface 223 of the piston rod 214 faces away from the inner small diameter pinion gear 227 of the drive control member 204.

The piston rod 214 comprises teeth 225. The teeth 225 extend along the main part 222 of the piston rod 214. In particular, the teeth 225 cover at least a part of the lower main surface 224 of the main part 222 of the piston rod 214. For example, the teeth 225 cover more than half of the lower main surface 224 of the main part 222 of the piston rod 214. The teeth 225 are adapted to engage the piston rod 214 with the inner small diameter pinion gear 227 of the drive control member 204. In particular, the teeth 225 are configured to prevent the piston rod 214 from moving, unless the drive control member 204 is enabled to rotate.

The spring member 213 comprises a coil spring. During assembly of the drive assembly 201, the spring member 213 is compressed between the first spring seat 261 and a second spring seat 262. The housing part 221 forms the second spring seat 262. A second end of the spring member 213 abuts the second spring seat 262, as shown in FIGS. 2 and 3.

Further, the spring member 213 is configured such that it is capable of delivering all the required doses from the cartridge 202 without being further compressed during a dose setting operation. In particular, in its compressed state, the spring member 213 exerts a force on the first spring seat 261 of the piston rod 214. Accordingly, when a locking of the piston rod 214 is released, this force tends to move the piston rod 214 in the distal direction 215. In particular, the spring member 213 exerts the force on the first spring seat 261 formed by the bearing 217 which moves the piston 218 in the distal direction 215 and results in expelling a medicinal product from the cartridge 202.

FIG. 5 shows the drive control member 204. The drive control member 204 runs on the shaft 266 which is an integral part of the dose setting member 203. For this purpose, the drive control member 204 comprises a through hole 226 in which the shaft 266 of the dose setting member 203 is arranged. The drive control member 204 comprises the inner small diameter pinion gear 227. The inner small diameter pinion gear 227 is located on an outer face 228 of the drive control member 204 which faces away from the dose setting member 203 in the assembled drive assembly 201. The inner small diameter pinion gear 227 is in toothed engagement with the piston rod 214, in particular with the teeth 225 of the piston rod 214.

Further, the drive control member 204 comprises teeth 229 located on its outer perimeter. The teeth 229 face in a direction away from the second axis 220. The teeth 229 arranged at the outer perimeter of the drive control member 204 are configured to engage with splines 230 on the actuator 212. The splines 230 in the actuator 212 are shown in FIG. 1. When the teeth 229 are engaged with the splines 230 of the actuator 212, the drive control member 204 is prevented from rotating relative to the actuator 212 and thereby also from rotating relative to the housing of the drug delivery device. However, if a user depresses the actuator 212, the drive control member 204 disengages from the actuator 212 and is enabled to rotate.

The drive control member 204 further comprises a set of crown gear teeth 231 which are arranged at its outer face 228 facing away from the dose setting member 203 in the assembled drive assembly 201. The set of crown gear teeth 231 is in permanent engagement with the reversing member 207.

The drive control member 204 also comprises a stop feature 232 which is configured to abut a corresponding stop feature 233 of the secondary drive control member 205 shown in FIG. 6 at the end of a dose dispense operation. The stop feature 232 of the drive control member 204 is arranged at an inner face 234 of the drive control member 204 being perpendicular to the second axis 220 and facing towards the dose setting member 203.

FIG. 6 shows the secondary drive control member 205. The secondary drive control member 205 is also mounted on the shaft 266 of the dose setting member 203. The secondary drive control member 205 comprises a through hole 235 wherein the shaft 266 extends through the through hole 235 in the assembled drive assembly 201. The outer face 236 of the secondary drive control member 205 facing away from the dose setting member 203 comprises the stop feature 233 which is configured to abut with the stop feature 232 of the drive control member 204 at the end of a dose dispense operation. An abutment of the stop feature 232 of the drive control member 204 and the stop feature 233 of the secondary drive control member 205 provides a rotational limit to a movement of the drive control member 204 at the end of a dose dispense operation.

Further, the secondary drive control member 205 comprises a perimeter surface 237 which faces away from the second axis in the assembled drive assembly 201. The perimeter surface 237 has a stepped form. In particular, the perimeter surface 237 has an inner area 238 and an outer area 239 wherein the inner area 238 has a slightly smaller diameter than the outer area 239.

On the perimeter surface 237 of the secondary drive control member 205, two sets of gear teeth 240, 241 are arranged. In particular, on the perimeter surface, an inner set of gear teeth 240 and an outer set of gear teeth 241 are arranged. The inner set of gear teeth 240 is arranged on the inner area 238 and the outer set of gear teeth 241 is arranged on the outer area 239.

The outer set of gear teeth 241 is releasably engaged with teeth 242 of the drive control member stop 206 shown in FIG. 1. This engagement causes the secondary drive control member 205 to be rotationally constrained, i.e. the secondary drive control member 205 is prevented from rotating relative to the drive control member stop 206 and thereby from rotating relative to the housing of the drug delivery device when the outer set of gear teeth 241 is engaged with teeth 242 of the drive control member stop 206.

The inner set of gear teeth 240 is configured to engage with the dose setting member 203 during dose dialing.

FIGS. 7 and 8 show perspective views of the dose setting member 203. The dose setting member 203 comprises the shaft 266 defining the second axis. In particular, the shaft 266 is integrally formed with the dose setting member 203. In a passive state, when no dose is set or dispensed, the dose setting member 203 is prevented from rotating relative to the housing, but can translate axially along the second axis 220. Further, the dose setting member 203 is permitted to rotate relative to the housing if it has previously been moved axially along the second axis, for example during the setting of a dose.

The dose setting member 203 further comprises an indicator 243 arranged at its outer surface facing away from the drive control member 204. On the indicator 243, dial numbers and graduations are printed. In particular, the housing comprises a pointer 252, which is shown in FIG. 13, wherein the pointer 252 points to one of the dial numbers or graduations, thereby indicating the number of a currently set dose. Accordingly, the dose setting member 203 is one of the elements of the drive assembly 201 allowing a user to control the operation of the drive assembly 201. In particular, the dose setting member 204 is used to set the intended dose and the indicator 243 of the dose setting member 204 comprising printed numbers and graduations is used to indicate by alignment with the pointer 252 attached to the housing the currently set dose.

The outer perimeter of the dose setting member 203 is held in the housing of the drug delivery device. In particular, the indicator 243 is held at its perimeter. Further, the axial translation of the dose setting member 203 is limited by stop features (not shown) on the housing of the drug delivery device and by the secondary drive control member 205.

Further, at an inner surface of the dose setting member 203 facing towards the secondary drive control member 205, gear features 244 are arranged. The gear features 244 of the dose setting member 203 provide a connection with the secondary drive control member 205 when the dose setting member 20 is translated axially during dose setting. In particular, the gear features 244 of the dose setting member 203 are configured to engage with the inner set of gear teeth 240 of the secondary drive control member 205.

The inner surface of the dose setting member 203 also acts on the drive control member stop 206 when translated axially during dose setting. In particular, the inner surface of the dose setting member 203 abuts the drive control member stop 206 such that the drive control member stop 206 follows an axial displacement of the dose setting member 203.

The drive control member 204 and the secondary drive control member 205 are located on the shaft 266 integrally formed by the dose setting member 203. Further, the coupling member 209 is rigidly fixed to an end 245 of the shaft 266. The end 245 of the shaft 266 has a non-circular cross-section rigidly fixing the coupling member 209 to the shaft 266.

The coupling member 209 comprises teeth 264. The teeth 264 may engage the reversing member 207. The reversing member 207 comprises teeth 265 arranged at its outer perimeter. The teeth 264 of the coupling member 209 may engage the teeth 265 of the reversing member 207.

As the coupling member 209 is rigidly fixed to the dose setting member 203, the coupling member 209 follows an axial movement of the dose setting member 203. Depending on the axial position of the dose setting member 203, the teeth 264 of the coupling member 209 are either engaged to the teeth 265 of the reversing member 207 or are arranged at a distance away from the teeth 265 of the reversing member 207.

When the teeth 264 of the coupling member 209 are engaged with the teeth 265 of the reversing member 207, a rotation of the coupling member 209 around the second axis 220 results in a rotation of the reversing member 207 around the main axis 219 and vice versa.

The drive control member stop 206 comprises teeth 242, as shown in FIG. 1. Further, the drive control member stop 206 is constrained at its outer surfaces in the housing such that it can only move in a direction that is parallel to the second axis 220. With no user input, the drive control member stop 206 is engaged with the secondary drive control member 205. In particular, the teeth 242 of the drive control member stop 206 are engaged with the inner set of gear teeth 240 of the secondary drive control member 205. Thereby, the secondary drive control member 205 is rotationally fixed to the housing.

An axial movement of the dose setting member 203 causes the drive control member stop 206 to disengage from the secondary drive control member 205, allowing the secondary drive control member 205 to rotate and a new dose end stop to be set.

The actuator 212, shown in FIG. 1, comprises a button 246 that may be pressed by a user. Further, the actuator 212 comprises a shaft 247. The shaft 247 and the button 246 are integrally formed. The shaft 247 extends from the button 246 in the direction parallel to the second axis 220 towards the dose setting member 203. The actuator 212 is constrained by the housing such that the actuator 212 can only move in a direction that is parallel to the second axis 220. In an alternative embodiment, the actuator may move in a direction which is inclined to the second axis 220. Further, splines 230 are arranged at the end of the shaft 247 facing away from the button 246. The splines 230 are engaged with the drive control member 204 when the button 246 is not depressed. This engagement prevents the drive control member from rotating relative to the actuator 212 and thereby from rotating relative to the housing of the drug delivery device. A depression of the button 246 causes the splines 230 to disengage from the drive control member 204. When disengaged from the splines 230, the drive control member 204 is enabled to rotate.

FIG. 9, as well as FIGS. 2 and 3, show the drive assembly 201 in a rest state. The rest state is a state before a dose setting operation is carried out.

The last dose stop drive member 211 comprises a set of gear teeth 248 which are engaged with the secondary drive control member 205. Accordingly, a rotation of the secondary drive control member 205 results in rotating the last dose stop drive member 211 relative to the housing.

Further, the last dose stop drive member 211 comprises a threaded portion 249. The last dose stop member 210 comprises a corresponding thread at its inner surface. The last dose stop member 210 runs on the threaded portion 249 of the last dose stop drive member 211. The last dose stop drive member 211 is constrained to the housing such that it can only rotate relative to the housing, but is prevented from moving axially along a linear axis parallel to the second axis 220 relative to the housing.

The last dose stop member 210 is threadedly engaged with the threaded portion 249 of the last dose stop drive member 211. The last dose stop member 210 is engaged by a spline feature 250 with the housing such that the last dose stop member 210 is prevented from rotating relative to the housing. Moreover, the last dose stop member 210 comprises a stop face. The stop face is configured to engage with the last dose stop drive member 211 when the permitted total number of doses has been selected.

In the rest state, the drive control member stop 206 is engaged with the secondary drive control member 205. Thereby, the secondary drive control member 205 is rotationally locked such that it can not rotate relative to the drive control member stop 206 or the housing of the drug delivery device.

Further, the splines 230 of the actuator 212 are engaged with the drive control member 204. Thereby, the drive control member 204 is rotationally locked such that it can not rotate relative to the actuator 212 and the housing of the drug delivery device. As the drive control member 204 is further engaged to the teeth 225 of the piston rod 214, the piston rod 214 is prevented from moving in a distal direction 215.

The stop feature 232 of the drive control member 204 is in abutment with the stop feature 233 of the secondary drive control member 205.

On the dose setting member 203, the “0” mark is in alignment with the pointer 252 of the housing.

The reversing member 207 is in toothed engagement with the drive control member 204 and the coupling member 209. In particular, the set of crown gear teeth 231 of the drive control member 207 are engaged with the teeth 265 of the reversing member 207. Further, the teeth 265 of the reversing member 207 are engaged with the teeth 264 of the coupling member 209.

As the drive control member 204 is prevented from rotating relative to the housing due to the engagement of the drive control member 204 with the splines 230 of the actuator 212, the coupling member 209 is also prevented from rotating relative to the housing. Thereby, the dose setting member 203 is prevented from rotating relative to the housing, as the coupling member 209 is further rigidly fixed to the end 245 of the shaft 266 of the dose setting member 203.

FIGS. 10 and 11 show the drive assembly 201 in a ready-to-set state. To enable dialing of a new dose, the dose setting member 203 has to be first pushed inwards in a direction along the second axis 220 by the user. Inwards means hereby that the dose setting member 203 is pushed towards the secondary drive control member 205.

When the dose setting member 203 is pushed inwards, this drives the drive control member stop 206 axially parallel to the second axis 220. Thereby, the drive control member stop 206 is disengaged from the secondary drive control member 205. Due to the disengagement from the drive control member stop 206, the secondary drive control member 205 is now allowed to rotate. Simultaneously, the secondary drive control member 205 engages the dose setting member 203 by an engagement of the inner set of gear teeth 240 of the secondary drive control member 205 engaging the gear features 244 of the dose setting member 203.

Moreover, in the ready-to-set state of the drive assembly 201, i.e. when the dose setting member 203 has been pushed inwards, the coupling member 209 being rigidly fixed to the dose setting member 203 is moved axially along the second axis 220 and is thereby disengaged from the reversing member 207. Due to the disengagement of the coupling member 209 from the reversing member 207, it is prevented that a rotation of the dose setting member 203 results in translating the piston rod 214.

However, as the coupling member 209 is disengaged from the reversing member 207 in the ready-to-set state, the coupling member 209 is now enabled to rotate relative to the housing. Thereby, the dose setting member 203 which is rigidly fixed to the coupling member 209 is also enabled to rotate relative to the housing in the ready-to-set state, i.e. after it has been pushed inwards.

Moreover, the drive control member stop 206 follows the axial movement of the dose setting member 203. Accordingly, in the ready-to-set state, the drive control member stop 206 abuts the splined end of the actuator 212, thereby preventing the actuator 212 from being moved axially in a direction towards the dose setting member 203. Accordingly, the actuator 212 cannot be depressed in the ready-to-set state.

Moreover, the drive control member 204 is prevented from rotating relative to the housing due to its engagement with the splines 230 of the actuator 212 in the ready-to-set state.

To set a new dose, a user rotates the dose setting member 203. The device may comprise a spring member (not shown) which may be connected to the dose setting member 203 in order to improve the ergonomics of the device.

FIG. 12 shows the drive assembly 201 in a dose-set state.

Compared to the ready-to-set state shown in FIGS. 10 and 11, the dose setting member 203 has been rotated. As the second drive control member 205 is now engaged to the dose setting member 203, the secondary drive control member 205 follows this rotation.

As there is no spring to compress during the dose setting operation, setting of the dose requires very little torque input.

In this new dose set position, the stop feature 233 of the secondary dose control member 205 has moved to provide a new end stop for the drive control member 204. The secondary drive control member 205 has been relocked in rotation by an engagement with the drive control member stop 206.

FIG. 13 shows a part of the housing 263 comprising a window 251.

As the dose setting member 203 has been rotated, the indicator 243 of the dose setting member 203 has been rotated as well. The set dose is now displayed on the indicator 243 of the dose setting member 203. The set dose can be viewed through the window 251 of the housing. Only a small group of printed numbers is visible through the window 251. A magnifying lens may be arranged in the window 251. Alternatively, the window may comprise a simple cutout in the housing. The pointer 252 on the housing points to the number corresponding to the set dose.

FIG. 14 shows the drive assembly 201 after the dose setting operation has been completed and before the dose dispense operation is initiated.

During dose setting, the drive control member 204 is rotationally fixed relative to the housing by its engagement to the actuator 212. The actuator 212 is configured such that the actuator 212 cannot be depressed while a dose setting operation is carried out. In particular, the drive control member stop 206 abuts the splined end of the shaft 247 of the actuator 212 such that the actuator 212 is prevented from moving in a direction along the second axis 220. Accordingly, a dose cannot be accidently delivered during dose setting as the dose delivery operation has to be initiated by depressing the actuator 212 which is prevented during dose setting.

After the dose setting operation has been completed, the user releases the dose setting member 203. The dose setting member 203 returns via a spring (not shown) to its original outward position, along with the drive control member stop 206. Now, the drive control member stop 206 does not abut the actuator 212 anymore such that the actuator is not locked against an axial movement anymore and can now be depressed by a user.

Moreover, before the actuator 212 is depressed by a user, i.e. before a dose dispensing operation is initiated, the set dose can be amended, i.e. it can be increased or decreased. Therefore, the user has to depress and rotate the dose setting member 203 again.

FIG. 15 shows the initiation of a dose dispensing operation. Further, FIG. 16 shows the drive assembly 201 during a dose dispensing operation.

In order to dispense a dose, the actuator 212 is pressed. This causes the actuator 212 to translate parallel to the second axis 220 and releases the splined connection between the actuator 212 and the drive control member 204. When the drive control member 204 is released, it is driven rotationally. In particular, the spring member 213 exerts a force on the piston rod 214. More particular, the spring member 213 exerts a force on the first spring seat 261 formed by the bearing 217 of the piston rod 214. As the drive control member 204 is not locked against a rotation anymore, the spring member 213 is enabled to expand. This results in a translation of the piston rod 214 in the distal direction 215. As the teeth 225 of the piston rod 214 are engaged to the inner small diameter pinion gear 227 (see FIG. 14) of the drive control member 204, the drive control member 204 is thereby rotated.

The axial translation of the piston rod 214 allows the bearing 217 to drive the piston 218 forward in a distal direction 215 further into the cartridge 202, thus delivering the dose of the medicinal product.

The drive control member 204 is rotated until its stop feature 232 reaches the new end stop position set by the stop feature 233 of the secondary drive control member 205. The end of the rotation of the drive control member 204 corresponds to the delivery of the dose being finished. When the stop feature 232 reaches the new end stop position, the drive control member 204 is prevented from rotating further relative to the housing. The engagement of the drive control member 204 with the piston rod 214 prevents a further translation of the piston rod 214 in the distal direction, thereby preventing the piston rod 213 from expelling more of the medicinal product from the cartridge 202.

During the dose dispensing operation, the indicator 243 of the dose setting member 203 automatically travels back to its “0” position such that “0′” is displayed in the window 251 of the housing. This is achieved by an interaction of the coupling member 209 and the reversing member 207. During dose dispense, the reversing member 207 is rotated due to its toothed engagement with the drive control member 204.

When the dose setting member 203 is moved outward to its original position after the dose setting has been completed and before the dose dispense is started, the coupling member 209 follows this movement as the coupling member 209 is rigidly fixed to the dose setting member 203. Thereby, the coupling member 209 engages the reversing member 207. Accordingly, the coupling member 209 is coupled via the reversing member 207 to the drive control member 204 during the dose dispense operation.

Further, the drive control member 204 is rotated during the dose dispense operation such that this rotation causes the coupling member 209, and hence the indicator 243, to rotate back to its zero display position.

The drug delivery device may comprise a feedback feature (not shown) which may indicate the end of a dispense operation by creating an audible click. Additionally or alternatively, the feedback feature may create an audible click with each unit dispensed. Additionally or alternatively, the feedback feature may create an audible feedback during the setting of a dose with each unit which is set.

Moreover, the drive assembly 201 comprises a last dose lockout assembly which is shown in FIG. 17. During dose setting, the secondary drive control member 205 rotates and this causes the last dose stop drive member 211 to rotate due to their toothed engagement. This in turn causes the last dose stop member 210, which is prevented from rotating, to translate along the longitudinal axis of the last dose stop drive member 211. During dose dispense, the secondary drive control member 205 does not rotate. Accordingly, the last dose stop drive member 211 also does not rotate.

When the maximum number of doses available has been dialed, the last dose stop member 210 reaches the end of the threaded portion 249 and the stop face of the last dose stop member 210 contacts a similar stop face on the last dose stop drive member 211. This prevents a further rotation of the last dose stop drive member 211. Thereby, also a further rotation of the secondary drive control member 205 and of the dose setting member 203 is prevented such that it is not possible to dial a larger dose. However, the number of units available for the last dose is now shown on the indicator 243 in the normal way before the final units are dispensed. This allows splitting the dose if required.

Furthermore, the drive assembly 201 comprises a safety member 253. FIG. 18 shows the drive assembly 201 comprising the safety member 253 in a state in wherein the drive assembly 201 is undamaged. FIG. 19 shows the drive assembly 201 comprising the safety member 253 in a state wherein the drive assembly 201 is damaged.

The safety member 253 is configured to prevent a movement of the piston rod 214 when the drive assembly 201 is damaged. The safety member 253 prevents the spring member 213 from automatically dispensing the remaining contents of the cartridge 202 when the drive assembly 201 is damaged, e.g. when the piston rod 214 is damaged.

The safety member 253 comprises a first safety member part 254 and a second safety member part 255. The first safety member part 254 comprises a strap 256. One end of the strap 256 is fixed to the bearing 217 of the piston rod 214 which corresponds to the first spring seat 261. The strap 256 runs parallel to the piston rod 214. In particular, the strap 256 is arranged to run along the upper main surface 223 of the piston rod 214.

The first safety member part 254 comprises a first engagement member 257 comprising teeth arranged on its surface facing away from the upper main surface 223 of the piston rod 214. The teeth of the first engagement member 257 extend along the same length as the teeth 225 of the piston rod 214.

The second safety member part 255 comprises a spring arm 258 which is attached to the housing part 221. The housing part 221 corresponds to the second spring seat 262. The spring arm 258 comprises a second engagement member 259 and a spacer member 260. The spacer member 260 abuts the piston rod 214 with a light spring force.

The second engagement member 259 is formed integrally with the spring arm 258. The second engagement member 259 comprises a protrusion which is configured to engage with the teeth of the first engagement member 257 of the first safety member part 254.

The strap 256 of the first safety member part 254 comprising the first engagement member 257 is connected to the first spring seat 261. Further, the second engagement member 259 of the second safety member part 255 is connected to the second spring seat 262. When the first and the second safety member parts 254, 255 are not engaged to each other in the undamaged state of the drive assembly 201, they do not provide a mechanical connection between the first and the second spring seat 262.

When the drive assembly 201 is undamaged, as shown in FIG. 18, the spacer member 260 holds the second engagement member 259 of the second safety member part 255 away from the first safety member part 254 by the tension of the piston rod 214.

Further, FIG. 19 shows a situation wherein the drive assembly 201 is damaged. This damage may result in the piston rod 214 releasing its tension.

For example, when the piston rod 214 breaks or is detached at either end, its tension loosens and the piston rod 214 becomes slack. In this condition, the spacer member 260 is enabled to overcome the now reduced tension of the piston rod 214. Accordingly, the spacer member 260 moves the piston rod 214 in a direction away from the first safety member part 254. This enables the first safety member part 254 to engage with the second safety member part 255. In particular, the teeth of the first engagement member 257 engage with the protrusion of the second engagement member 259.

The engagement of the first and the second safety member parts 254, 255 locks the spring member 213. In particular, the engagement of the first and the second safety member parts 254, 255 fixes the distance between the first and the second spring seat 262 such that the first and the second spring seats 261, 262 are prevented from moving relative to each other, as the first safety member part 254 is fixed to the first spring seat 261 formed by the bearing 217 and the second safety member part 255 is fixed to the second spring seat 262 formed by the housing part 221. When the distance between the spring seats 261, 262 is fixed, the spring member 213 is prevented from relaxing any further.

In particular, the first safety member part 254 is now prevented from moving in the distal direction 215 any further as it is engaged to the housing part 221 via the second safety member part 255. As the first safety member part 254 is fixed to the first spring seat 261 at one end, the first spring seat 261 can not move in the distal direction 215 when the first and the second safety member parts 254, 255 are engaged to each other. This prevents a further movement of the spring member 213 and thereby of the piston rod 214. Accordingly, a further dose dispensing is also prevented.

In other words, the first safety member part 254 and the second safety member part 255 are constructed such that they mechanically engage with each other only in case the piston rod 214 releases its tension, i.e. in case the drive assembly 201 is damaged. The damage of the drive assembly 201 is detected mechanically. In particular, the damage of the drive assembly 201 is detected by the mechanical engagement of the first safety member part 254 and the second safety member part 255. When the damage of the drive assembly 201 is detected mechanically, the safety member 253 prevents a further movement of the piston rod 214, thereby preventing a further dose dispensing.

### REFERENCE NUMERALS

201 drive assembly
202 cartridge
203 dose setting member
204 drive control member
205 secondary drive control member
206 drive control member stop 206
207 reversing member
208 reversing member shaft
209 coupling member
210 last dose stop member
211 last dose stop drive member
212 actuator
213 spring member
214 piston rod
215 distal direction
216 proximal direction
217 bearing
218 piston
219 main axis
220 second axis
221 housing part
222 main part
223 upper main surface
224 lower main surface
225 teeth of the piston rod
226 through hole
227 inner small diameter pinion gear
228 outer face
229 teeth
230 splines
231 set of crown gear teeth
232 stop feature
233 stop feature
234 inner face
235 through hole
236 outer face
237 perimeter surface
238 inner area
239 outer area
240 inner set of gear teeth
241 outer set of gear teeth
242 teeth of the drive control member stop
243 indicator
244 gear features
245 end of the shaft 266
246 button
247 shaft
248 set of gear teeth
249 threaded portion
250 spline feature
251 window
252 pointer
253 safety member
254 first safety member part
255 second safety member part
256 strap
257 first engagement member
258 spring arm
259 second engagement member
260 spacer member
261 first spring seat
262 second spring seat
263 housing
264 teeth of the reversing member
265 teeth of the coupling member
266 shaft of dose setting member

1. A drive assembly (201) for a drug delivery device, comprising
a piston rod (214) comprising a bearing (217) at its distal end, and
a safety member (253) that is configured to prevent a movement of the bearing (217) of the piston rod (214) when the drive assembly (201) is damaged.

2. The drive assembly (201) according to claim 1,
wherein the piston rod (214) is tensed in an undamaged state of the drive assembly (201), and
wherein the drive assembly (201) being damaged results in the tension of the piston rod (214) being relieved.

3. The drive assembly (201) according to one of the preceding claims,
wherein the safety member (253) comprises a first safety member part (254) and a second safety member part (255) which is engageable to the first safety member part (254) and
wherein a movement of the bearing (217) is prevented when the first safety member part (254) and the second safety member part (255) are engaged with each other.

4. The drive assembly (201) according to claim 3,
wherein the safety member (253) comprises a spacer member (260) that is adapted to prevent an engagement of the first and the second safety member part (255) when the drive assembly (201) is undamaged.

5. The drive assembly (201) according to claim 4,
wherein the spacer member (260) abuts the piston rod (214).

6. The drive assembly (201) according to one of claims 2-5,
comprising a spring member (213),
wherein the drive assembly (201) is configured such that a relaxation of the spring member (213) moves the piston rod (214) and wherein an engagement of the safety member parts (254, 255) prevents the relaxation of the spring member (213).

7. The drive assembly (201) according to claim 6,
wherein the spring member (213) is tensed between a first spring seat (261) and a second spring seat (262), and
wherein the first safety member part (254) is connected to the first spring seat (261) and the second safety member part (255) is connected to the second spring seat (262).

8. The drive assembly (201) according to claim 6,
wherein an engagement of the first safety member part (254) and the second safety member part (255) prevents a movement of the first spring seat (261) relative to the second spring seat (262), thereby preventing further relaxation of the spring member (213).

9. The drive assembly (201) according to one of claims 2-8,
wherein the first safety member part (254) comprises a first engagement member (257) comprising teeth arranged at a surface of the first safety member part (254).

10. The drive assembly (201) according to claim 9,
wherein the second safety member part (255) comprises a second engagement member (259) comprising a protrusion which is adapted to engage with the first engagement member (257).

11. The drive assembly (201) according to one of claims 2-10,
wherein one of the first or the second safety member parts (254, 255) is attached to the piston rod (214).

12. The drive assembly (201) according to one of claims 2-11,
wherein one of the first or the second safety member parts (254, 255) is attached to a housing of the drug delivery device.

13. The drive assembly (201) according to one of the preceding claims,
wherein the piston rod (214) is flexible.

14. The drive assembly (201) according to one of the preceding claims,
wherein a part of the piston rod (214) is wound around a drive control member (203).

15. The drive assembly (201) according to one of the preceding claims,
wherein the safety member (253) is configured to mechanically detect a damage of the drive assembly (201).

16. The drive assembly (201) according to one of the preceding claims,
wherein the drive assembly (201) is a manually operable assembly.

17. A drug delivery device comprising a drive assembly (201) according to one of the preceding claims.
